(19) United States
(12) Patent Application Publication
(10) Pub. No.: US 2012/0053419 A1
(43) Pub. Date: Mar. 1, 2012

(54) Highly Articulable Catheter

(76) Inventor: Eliot BLOOM, Hopkinton, NH (US)

(21) Appl. No.: 12/862,677

(22) Filed: Aug. 24, 2010

(51) Int. Cl. A61B 1/01; A61B 5/00; A61B 10/02; A61M 25/092; A61B 1/07

(52) U.S. Cl. 600/146; 604/95.04; 600/562; 600/573; 600/104

(57) ABSTRACT

A flexible highly-articulable dual-catheter device includes an outer catheter and an inner catheter. The outer catheter contains at least one lumen to support advancement and in-situ replacement of the inner catheter. An optional second lumen in the outer catheter enables carriage of a fiber-optic line for vision to aid a clinician to advance the dual-catheter device to the desired location in a body. The inner catheter is a multi-lumen catheter that enables a hypotube and a navigation wire to be passed through two of the lumens. The provision of a multi-segment region placed at the distal end of the hypotube, with each segment of a differing durometer, enables a wide range of articulability of the device. An optional second navigation wire can also be coupled to the distal end of the hypotube to provide additional articulation. Both diagnostic and therapeutic tools can be coupled to the elongated device. Various tips such as RF energy and aspiration tips can be coupled to the dual-catheter device.

### BACKGROUND

1. Field

The present invention relates to an elongated medical device for insertion into a tortuous pathway in a body, and in particular to a catheter device for use in diagnostic and therapeutic applications.

2. Background Art

Medicine is providing ever-increasing demands for devices that can navigate narrow passageways to a desired location so that diagnostic and therapeutic procedures can be performed at that location. Currently, elongated medical devices such as catheters can extend from outside a body via an access point through various connected passageways to the desired location. In these elongated medical devices, one or more lumens are provided by which medical tools and sensors can be introduced to the desired location and by which fluids and/or tissues can be delivered to and/or sampled and aspirated from the desired location.

Such elongated medical devices must meet a wide variety of requirements in order to provide their desired functionality. For example, these devices must provide the required length to reach the desired location, yet have an outer diameter small enough in diameter to traverse the narrow passageways but an inner diameter sufficiently large enough to provide the required functionality in this device. In addition, in order to reach the desired location, the elongated medical device must have ample longitudinal strength so that a clinician can advance the device the entire distance to the desired location. The longitudinal strength is important since when a pushing force is applied to the proximal end of an elongated medical device, an equal movement should be transmitted to the distal end of the device. Further, the device must also possess sufficient flexibility to be able to navigate the bends and angles presented by the passageways without undergoing a catastrophic collapse or fracture such as kinking. Finally, the device must also support sufficient torqueability such that a tool located at the end of the device can be rotated to a desired position or orientation. In summary, the device needs to meet the requirements of pushability, torqueability and flexibility.

These design considerations translate into competing design requirements. In particular, construction of the elongated device must compromise between outer diameter, torqueability, strength and flexibility. For example, a requirement that an elongated medical device be capable of traversal of the adult bronchial tree to the 5th branch would require an outer diameter of less than or equal to about 5 mm as well as extraordinary flexibility and a wide range of articulability. The strength and torqueability requirements of such a bronchial catheter, however, suggest a stronger, more robust shaft design that would have a larger diameter and be less flexible.

In addition to the compromise required for navigation and articulation through the narrow passageways, it is also desirable that a means be provided by which a clinician can view the progress and immediate surroundings of the distal end of the device. Further, it is desirable that a variety of tools, both diagnostic and therapeutic, be capable of being interchangeably used with the medical device while the distal end of the device maintains its position at the desirable location in the body.

### BRIEF SUMMARY

What is needed is an elongated medical device that can navigate a tortuous pathway within a body in a highly aritculable fashion. In a particular embodiment, it is desirable that an elongated medical device be provided with two catheters, with the outer diameter of the outer catheter being less than or equal to about 5 mm.

In an embodiment of the present invention, a dual-catheter apparatus is provided that contains an outer catheter having at least one lumen, and an inner catheter that is disposed within that lumen. The inner catheter has a distal end to which a tool (e.g., a diagnostic or therapeutic tool) is connected. A navigation wire (e.g., a tether) is coupled to the distal end of the inner catheter so that manipulation of the navigation wire causes deflection of the distal end of the inner catheter, thereby providing steerability of the dual-catheter apparatus.

In a further embodiment of the present invention, a dual-catheter apparatus provides vision to the clinician using a fiber-optic device at the distal end of the dual-catheter apparatus. The fiber-optic device is connected through the dual-catheter apparatus via a fiber-optic lumen in the outer catheter. Illumination of the distal end of the dual-catheter apparatus allows a clinician to view progress of the advancement of the dual-catheter apparatus along the tortuous pathway within the body.

In a still further embodiment of the present invention, the outer surface of the inner catheter is coated with a hydrophilic layer so that the inner catheter can move independently of the outer catheter. Such a layer enables the inner catheter to be extended beyond the outer catheter at a desired location within the body. Such a layer also allows one inner catheter to be interchanged with another inner catheter while the outer catheter remains substantially stationary within the tortuous pathway within the body. Another embodiment has the inner surface of the outer catheter coated with a hydrophilic layer. In another embodiment, a lubricant is disposed between the inner surface of the outer catheter and the outer surface of the inner catheter. Either inner catheter or outer catheter can therefore be used as a working channel in an embodiment of the present invention. For example, once positioned within a body, the inner catheter can be removed and the lumen of the outer catheter can be used as a pathway to deliver another catheter or tool into the body. Similarly, the outer catheter can be removed and the inner catheter can be used like a guide wire with another catheter or tool advanced over the inner catheter into the body.

In yet another embodiment of the present invention, the inner catheter contains a hypotube connected to a multi-segment distal tip. Adjoining segments in the multi-segment distal tip are of different lengths and different durometers to support various ranges of articulation in response to manipulation by the navigation wire.

In a still further embodiment of the present invention, a second navigation wire can be attached to the distal end of the inner catheter, but at an orthogonal point to the first navigation wire, so that two orthogonal ranges of deflection are available to the clinician for enhanced steerability.

In another embodiment of the present invention, the inner catheter contains a hypotube with transverse slot patterns fabricated into the distal end of the hypotube. Such transverse slot patterns can be designed to provide a desired degree of flexibility, pushability and torqueability.

Further embodiments, features, and advantages of the invention, as well as the structure and operation of the various embodiments of the invention are described in detail below with reference to the accompanying drawings.

### BRIEF DESCRIPTION OF THE DRAWINGS/FIGURES

Embodiments of the present invention are described with reference to the accompanying drawings. In the drawings, like reference numbers indicate identical or functionally similar elements.

FIG. 1 illustrates an elongated medical device, in accordance with an embodiment of the present invention.

FIG. 2 illustrates a dual-catheter device, in accordance with an embodiment of the present invention.

FIG. 3 illustrates a cross-section of an outer catheter of a dual-catheter device, in accordance with an embodiment of the present invention.

FIG. 4 illustrates a cross-section of another embodiment of the outer catheter of a dual-catheter device, in accordance with an embodiment of the present invention.

FIG. 5 illustrates a cross-section of an outer catheter of a dual-catheter device, in accordance with another embodiment of the present invention.

FIG. 6 illustrates a cross-section an embodiment of a dual-catheter device illustrating an inner catheter 620 disposed in a lumen 320 of an outer catheter 210, in accordance with an embodiment of the present invention.

FIG. 7 illustrates a hypotube that forms a part of an inner catheter, in accordance with an embodiment of the present invention.

FIG. 8 illustrates a flat view of an exemplary machining pattern in a hypotube, in accordance with an embodiment of the present invention.

FIGS. 9A and 9B illustrate a distal tip 760 of the hypotube of FIG. 7 with either one or two navigation wires, in accordance with embodiments of the present invention.

FIG. 10 illustrates a cross-section of another embodiment of a dual-catheter device, in accordance with an embodiment of the present invention.

FIG. 11 provides a flowchart of a method for navigating a tortuous pathway in a body using a dual-catheter device, according to an embodiment of the current invention.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

This specification discloses one or more embodiments that incorporate the features of this invention. The disclosed embodiment(s) merely exemplify the invention. The scope of the invention is not limited to the disclosed embodiment(s). The invention is defined by the claims appended hereto.

The embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

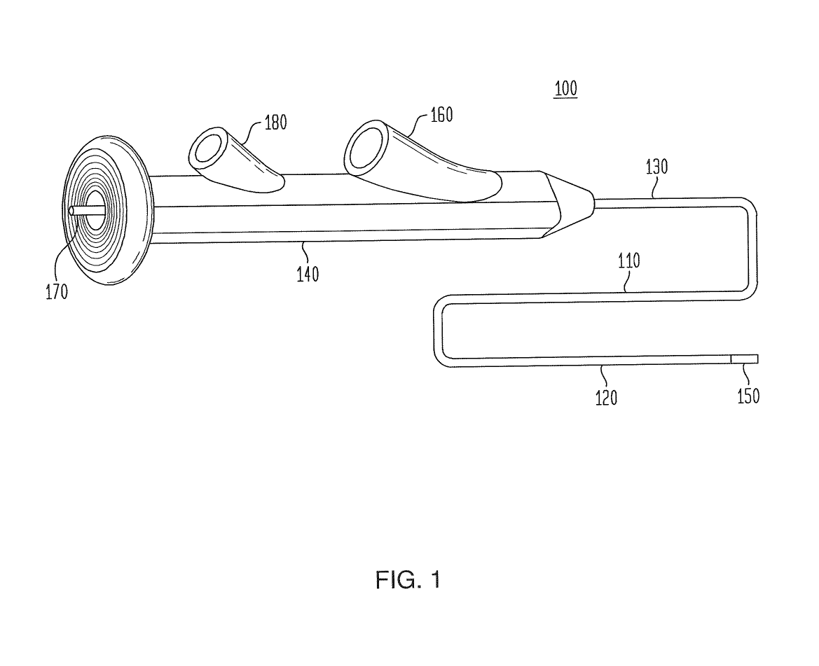

FIG. 1 depicts an embodiment of the articulable catheter 100, in accordance with an embodiment of the present invention. Articulable catheter 100 contains an elongated shaft 110 having a distal end 120 and a proximal end 130. Connected to proximal end 130 is a handle 140. Connected to distal end 120 is an exposed end 150. In various embodiments, exposed end 150 connects to a variety of tools such as diagnostic and therapeutic tools that can operate at a desired location within the body of a living organism such as a human or animal. Diagnostic tools include assorted biopsy tools and devices, and therapeutic tools include advanced-energy and pharmaceutical tools. Based on the location within the body for which access is sought, elongated shaft 110 can take on a wide variety of lengths. Ports 160, 170 and 180 provide access to one or more lumens of catheter 100 to permit passage of other catheters or instruments (e.g., power to a connected tool, a vision system (e.g., fiber-optic device), an aspiration needle, a drug-delivery catheter, a biopsy instrument, a cutter, a balloon catheter, a electrocautery instrument, a hemostatic sealing instrument, etcetera).

As noted above, flexibility is a requirement imposed on elongated medical devices such as catheters. In the context of the present invention, articulable catheter 100 must have sufficient flexibility to meet the particular application for which it is being used. That is, the degree of flexibility required to navigate a particular tortuous passageway is dependent upon the pathway's particular twists and bends, and the curvature thereof. Tighter twists and bends demand greater flexibility than more gentle twists or bends. Accordingly, the flexibility for embodiments of articulable catheter 100 is application dependent. For example, a bronchoscope embodiment of the present invention can require greater flexibility to navigate to the 5th branch of an adult bronchi system that would be required for a catheter to navigate a more modest pathway in the body. As described herein, flexibility is controlled by the dimensions of the inner and outer catheters, the durometers of the materials used in their construction, and on the dimensions, materials and construction of the hypotube.

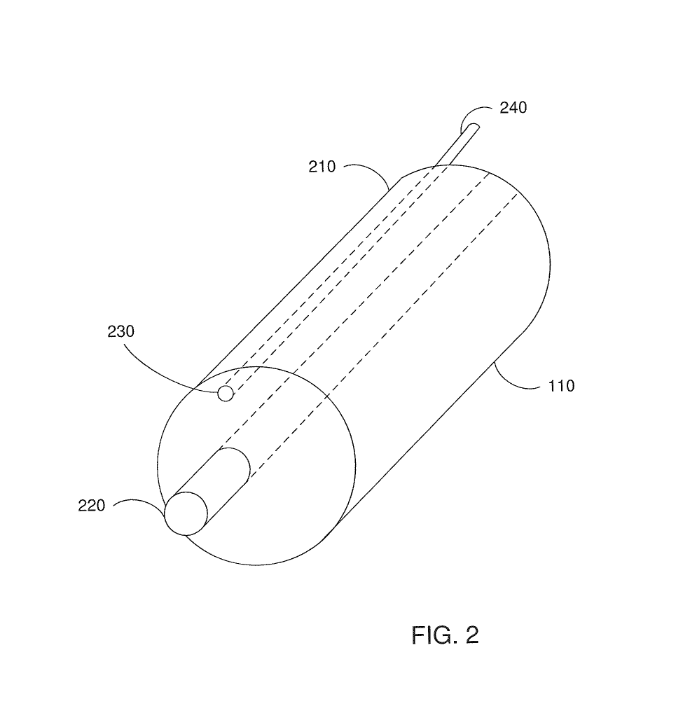

FIG. 2 depicts a perspective view of a section of elongated shaft 110 that illustrates the dual-catheter configuration, in accordance with an embodiment of the present invention. Outer catheter 210 and inner catheter 220 are shown. A fiber-optic lumen 230 is configured to accept a fiber-optic line 240. In addition, fiber-optic lumen 230 can optionally support fluid introduction for lens cleaning of the fiber-optic system (e.g., a lens or camera system) located at distal end 120.

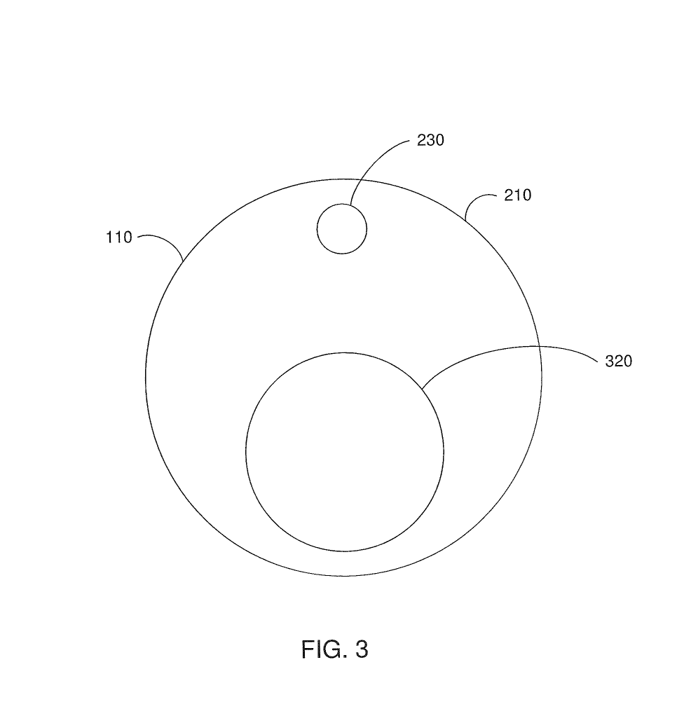

FIG. 3 depicts a transverse cross-sectional view of elongated shaft 110 that illustrates the dual-catheter configuration of outer catheter 210 shown in FIG. 2. Outer catheter 210 and lumen 320 for inner catheter 220 (not shown) are illustrated. Also shown is fiber-optic lumen 230 for the optional delivery of fiber-optics to distal end 120. As discussed below, inner catheter 220 provides the steerability necessary to navigate elongated shaft 110 through passageways to the desired location in the body. As FIG. 3 illustrates, lumen 320 can be eccentric with respect to a central longitudinal axis of outer catheter 210. In an alternate embodiment of the present invention, lumen 320 can be concentric to the central longitudinal axis of outer catheter 210. The eccentric embodiment provides an advantage for certain tools that are connected to inner catheter 220. For example, a coring tool connected to inner catheter can core a greater diameter of tissue than the diameter of inner catheter 220 by rotation of outer catheter 210. When inner catheter 220 is eccentric to outer catheter 210, rotation of outer catheter 210 sweeps out an area that exceeds that of the cross-section of inner catheter 220. Thus, a coring tool attached to inner catheter 220 having a diameter of 2.7 mm can core a diameter of approximately 4.0 mm, i.e., roughly the size of outer catheter 210 which has a diameter of 4.2 mm.

In one embodiment of the eccentric version, outer catheter 210 has a diameter of approximately 4.2 mm. Fiber-optic lumen 230 has a diameter of approximately 0.81 mm and is located proximate to the peripheral wall of outer catheter 210. Lumen 320 is also located proximate to the peripheral wall of outer catheter 210 (approximately 0.025 mm away), but diametrically opposite to the location of fiber-optic lumen 230. Lumen 320 has a diameter of approximately 3.4 mm. Within lumen 320, an inner catheter 220 having a diameter of approximately 2.7 mm can be disposed. These dimensions and eccentric configurations are examples of various configurations of the dual-catheter device, and are not limiting to the device. Other dimensions and configurations that support the particular tools and specific navigation to a desired location inside the body are within the scope of the present invention.

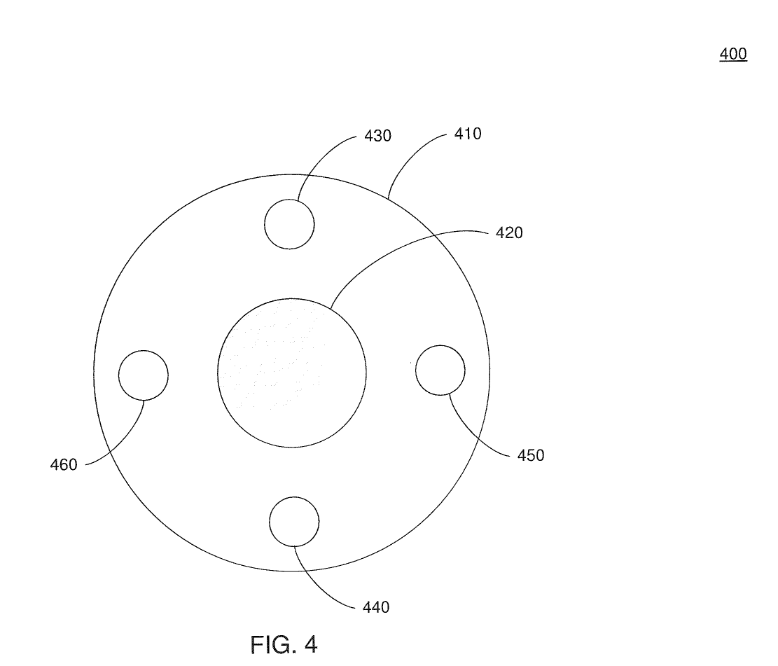

FIG. 4 illustrates an additional embodiment of an outer catheter 410 of a multi-lumen dual-catheter system 400. In this example embodiment, outer catheter 410 includes four peripheral lumens 430, 440, 450 and 460, together with a central lumen 420 for inner catheter 220. One of the four peripheral lumens, e.g., lumen 430 can provide for the passage of the fiber-optic system, as described earlier. Each of the other three peripheral lumens 440, 450 and 460 can be used, for example, for additional articulation, introduction of fluids and aspiration, etcetera, as required. Although FIG. 4 illustrates the lumen configuration as being symmetric and concentric, asymmetric and eccentric lumen configurations also fall within the spirit of the present invention. In particular, lumens 430 through 460 can be of different physical dimensions and placed in a variety of different physical locations consistent with being located within outer catheter 410.

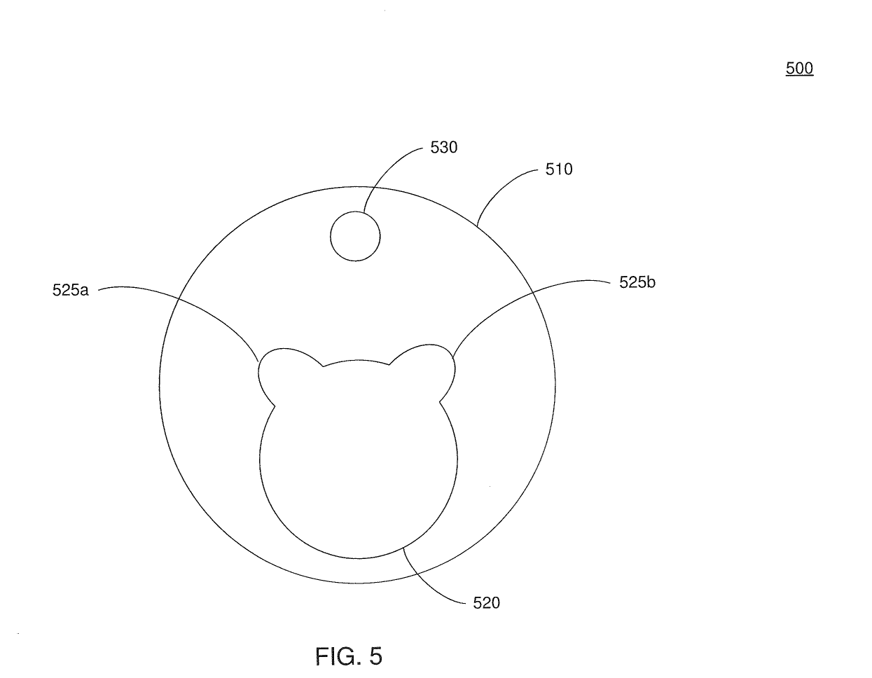

Yet another example embodiment of an outer catheter 510 of a dual-catheter system 500 is shown in FIG. 5. As illustrated in FIG. 5, a lumen 520 for advancement of inner catheter 220 need not be completely circular in shape, but can be configured to provide extensions beyond a circular shape. Shrinkage resulting from manufacture of lumen 320 within outer catheter 210 can result in a distortion of the nominally circular cross-section of lumen 320. Such distortion can be attributed to the significant difference in material in the top half compared to the bottom half of outer catheter 510. By extending the shape of lumen 520 beyond a circular perimeter in the manner illustrated by ears 525a, 525b, the possibility of an egg-shaped lumen resulting from shrinkage can be avoided. An egg-shaped lumen can pose significant challenges to the advancement and alignment of inner catheter 220. Such desired shape extensions of lumen 320 are particularly useful when inner catheter 220 is disposed close to one side of the peripheral wall of outer catheter 210, as illustrated in FIG. 3. The particular shape extensions 525a, 525b are exemplary only and are not limiting. Any shape extensions of lumen 520 beyond a circular perimeter to offset shrinkage forces fall within the scope of the present invention.

With respect to materials, it is desirable that the material used for the fabrication of outer catheter 210 provide an appropriate compromise between strength, flexibility and other requirements. For example, polymers with high hardness or durometer can meet the longitudinal strength or stiffness requirements, while materials with low hardness or durometer can meet the flexibility requirements. Materials that provide the appropriate compromise between these two extremes include silicones, polyurethane, elastomeric polyamides, block polyamide (such as Pebax®, a polyether block amide, available from Arkema, Colombes, France), Tecoflex® and various co-polymers. The range of durometers suitable for the manufacture of outer catheter 210 include durometers in the range 20 to 70 Shore A.

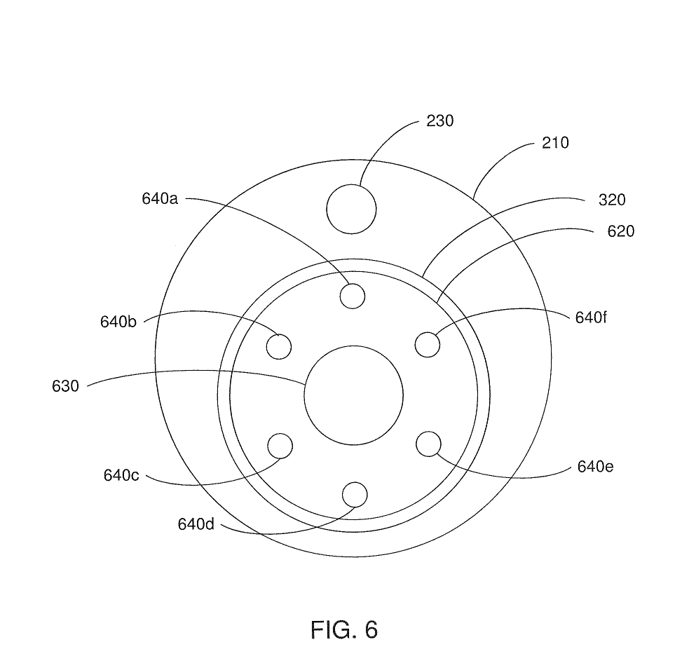

FIG. 6 illustrates an embodiment 620 of inner catheter 220 shown disposed in lumen 320 of outer catheter 210. In this embodiment, inner catheter 620 includes seven lumens 630 and 640a through 640f. One configuration of those seven lumens 630 and 640a through 640f is shown in FIG. 6, where an inner lumen 630 is provided for disposition of inner catheter 620, while at least one of the remaining lumens 640a through 640f, e.g., 640d, is used for articulation. Articulation lumen 640d provides a passageway for a navigation wire (e.g., a tether) that provides means for articulation of inner catheter 620. The remaining lumens 640a, 640b, 640c, 640e, 640f can be used for delivery of a tool (either diagnostic or therapeutic), to provide additional articulation of inner catheter 620 through a further navigation wire, or to deliver or receive a tissue or fluid. For example, connectivity to a therapeutic tool can include a 4-wire electrical connection, when the connected therapeutic tool is an RF-based coring tool. The number of wires can be determined by the amount of power needed to support the functionality of the tool, as well as to provide mechanical stability of the tool. Mechanical stability of a tool typically requires at least three wires in order to limit unintended motion in all three dimensions. Other tools that require connectivity can use one or more of remaining lumens 640a, 640b, 640c, 640e, 640f, as required. One of remaining lumens 640a, 640b, 640c, 640e, 640f can be used to provide additional degrees of freedom of articulation that exceed the single degree of freedom provided by the single navigation wire discussed above. Although FIG. 6 illustrates the inner catheter configuration as being symmetric and concentric, asymmetric and eccentric configurations also fall within the spirit of the present invention. In particular, lumens 630 and 640a through 640f can be of different physical dimensions and placed in a variety of different physical locations consistent with being located within inner catheter 620. Inner catheter 620 is coated with a hydrophilic layer to provide a slippery surface. A hydrophilic layer contains a chemical that combines with water molecules to offer a low friction surface when water is applied. Examples of hydrophilic coatings include polyethylene glycol, polyvinyl alcohol, polyisopropyl allylamide and polyvinyl pyrrolidinone. In an exemplary embodiment of inner catheter 620, it is fabricated from a block polyamide such as Pebax®, and has a 2.7 mm outer diameter. Lumen 630 has a diameter of approximately 1.5 mm, and lumens 640a through 640f have diameters that typically are in the range of 0.2 mm to 0.4 mm. Such a hydrophilic layer enables inner catheter 620 and outer catheter 210 to move independently of one another. Such independence is desirable when navigating articulable catheter 100 around bends in a tortuous pathway, where the outer radius of the bend exceeds the inner radius of the bend. In addition, when articulable catheter 100 reaches a desired location in a body, it is also desirable to extend inner catheter 620 (with tool attached) independently of outer catheter 210 at that location. As an alternative to a hydrophilic layer being applied to the outer surface of the inner catheter (as described above), the hydrophilic layer can instead (or additionally) be applied to the inner surface of the outer catheter. As a further alternative, a lubricant (other than a hydrophilic layer) can be disposed between the inner surface of the outer catheter and the outer surface of the inner catheter. Either inner catheter or outer catheter can therefore be used as a working channel in an embodiment of the present invention.

In an embodiment of the present invention, inner catheter 620 can be readily replaced with another inner catheter. Such an embodiment enables, for example, replacement of a diagnostic tool by a therapeutic tool, while outer catheter 210 remains situated at the desired location within the body. To ensure ease of insertion and removal, inner catheter 620 can be coated with the hydrophilic layer mentioned above. Alternatively, the outer catheter can be removed and the inner catheter can be used like a guide wire with another catheter or tool advanced over the inner catheter into the body

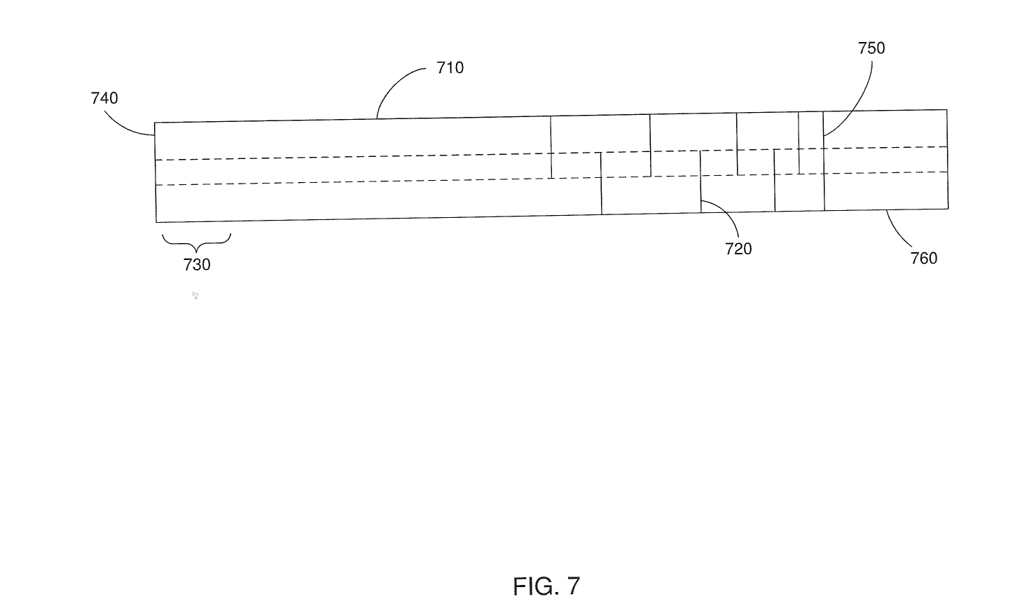

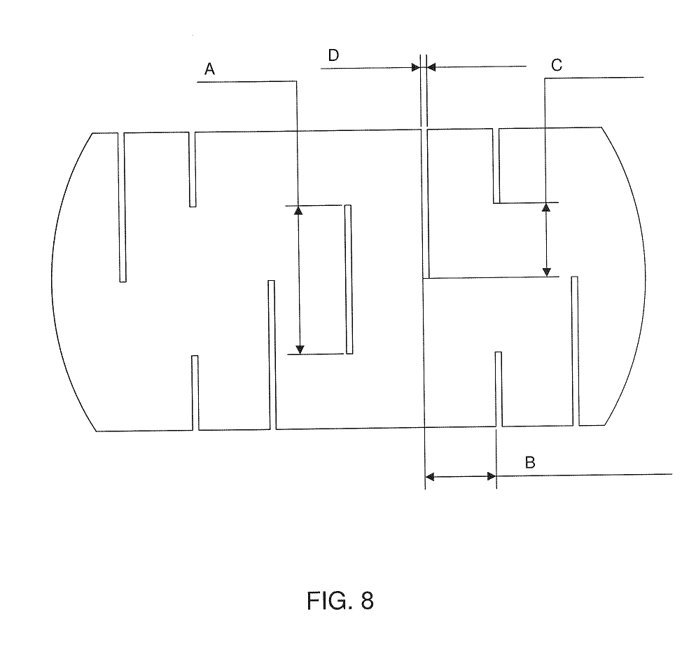

In one embodiment, an inner tube can be placed within inner lumen 630 consistent with the requirements of pushability, torqueability and flexibility. In an exemplary embodiment of the inner tube, FIG. 7 illustrates a hypotube 710 that includes a cutting pattern 720 formed within hypotube 710. The term hypotube refers to a long shaft or tube in a catheter or needle device that is used to deliver an attached device into the body of a living organism such as a human or animal. Hypotube 710 has a proximal region 730 defining a proximal end 740 and a distal end 750. Coupled to distal end 750 of hypotube 710 is distal tip 760. Hypotube 710 can be formed of a metallic material such as stainless steel that meets the strength, flexibility and torqueability requirements of the tortuous pathway to be encountered. Hypotube 710 can be formed having any desired length, width and material thickness as required to satisfy the requirements of any particular elongated device application. Hypotube 710 provides the opportunity for variable characteristics (e.g., flexibility) along its length, in contrast to alternative approaches such as a braid. Braid features have a fixed pitch and are unable to readily provide such variable characteristics. In an exemplary embodiment of hypotube 710, approximately 75 cm of its length adjacent to distal end 750 includes a cutting pattern 720, while 50 cm of its length adjacent to proximal end 740 does not include a cutting pattern 720. Cutting pattern 720 can be formed using any suitable technique, such as laser machining. Choice of the pattern in cutting pattern 720 can be determined based on the desired longitudinal strength and flexibility. In order to provide the required flexibility (or strain relief), the pattern of cutting pattern 720 can be varied over the length of hypotube 710. In an exemplary embodiment of the pattern of cutting pattern 720, transverse slots are cut into hypotube 710, with slot lengths varied over the length of hypotube 710. For example, a greater frequency of transverse slots can be positioned close to distal end 750, with fewer transverse slots positioned further away from distal end 750. In an exemplary embodiment of hypotube 710, the particular steel used is stainless steel series 3, the outer and inner diameter are 1.68 mm and 1.52 mm respectively, and the transverse slots have the following pattern shown in a flat view in FIG. 8. Dimensions of the exemplary embodiment of hypotube 710 are A=2.59 mm, B=1.52 mm, C=1.30 mm and D=0.10 mm. Such a pattern can be used for 76 cm of length of hypotube 710 closest to its distal end, while 50 cm of hypotube 710 closest to its proximal end can be without a cutting pattern.

In a further embodiment of the present invention, a vision-delivery mechanism can be provided to deliver illumination to the end of the dual-catheter device and return vision (e.g., optical images) from that location. For example, fiber optics can be used to provide a light-guided, dual-catheter embodiment. Referring to FIGS. 1 and 2, a fiber-optic line 240 is disposed within fiber-optic lumen 230 of outer catheter 210. In this embodiment, a fiber-optic device (not shown) containing a light-radiating structure extends from exposed end 150 of elongated shaft 110 to slightly protrude outward from exposed end 150 into the immediate vicinity of the body. Such a protrusion ensures that during navigation, extraneous material (e.g., mucous) can be wiped away from the fiber-optic device during motion of elongated shaft 110 while being navigated in the tortuous pathway of the body. Reflected light from such illumination is provided to a vision system (not shown) co-located with light producing device. For example, a vision system available from Myriad Fiber Imaging, Dudley, Mass. can be used. Such illumination can be used while advancing the dual-catheter device within the body in order that the clinician can see ahead of exposed end 150 of elongated shaft 110 when navigating, and in particular, when navigating a bend or curve in the tortuous pathways encountered. Using such illumination while advancing elongated shaft 110, the clinician can obtain direct visual confirmation that elongated shaft 110 is proceeding smoothly through junctions and past bends (particularly acute bends) without complications. Choice of wavelengths of light are based on the particular diagnostic and therapeutic operation that needs to be performed, as well as the particular location within the body for which the procedure is desired. Thus, differences in the penetration and scattering of wavelengths through the body tissues of interest ultimately determine the particular choice of optical wavelength. For example, red wavelengths and green wavelengths can be used, but any wavelength can be used with embodiments of the present invention to satisfy particular optical requirements imposed by the targeted tissue location and the accuracy required of the viewing area. In addition, the optical power levels are also chosen based on the same optical requirements. The fiber-optic device can be operated continuously, intermittently, or in a pulsed mode in order to optimize the visual picture provided to the clinician. The fiber-optic device can be attached at the distal end of the outer catheter by use of a reflow collar or other suitable means. Finally, the fiber-optic device (and more generally the vision system) can be replaceable and can be re-used after appropriate sterilization in another outer catheter 210 for a subsequent procedure. Such an option improves the cost effectiveness of the apparatus when the vision system is a relatively expensive component. Withdrawal of the vision system can be effected by a manipulation of the vision system at the distal end to remove the attachment mechanism, with the vision system withdrawn through the proximal end of outer catheter 210. Following sterilization, the previously used vision system can then be re-installed in outer catheter 210 via its proximal end. In additional embodiments, outer catheter 210 can be completely disposable after one use, or can be re-usable over the life cycle of the vision system. In a further embodiment, the entire dual-catheter device can be disposable.

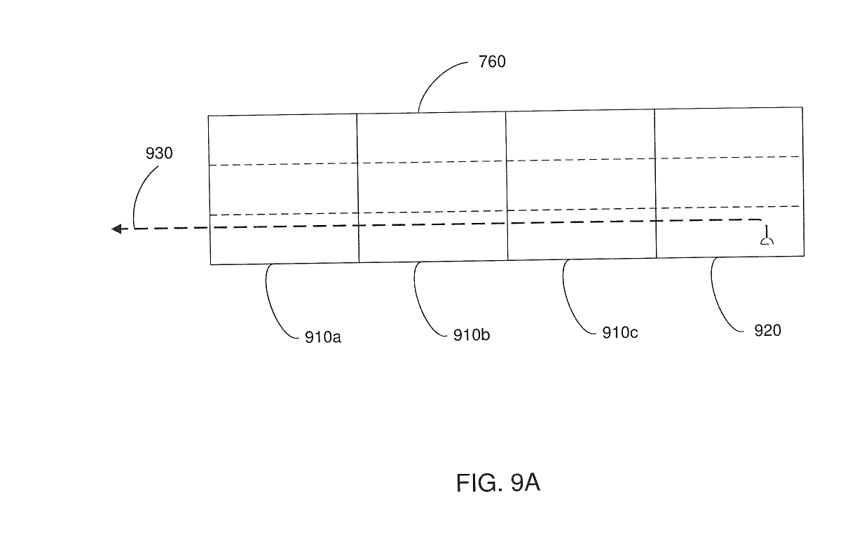

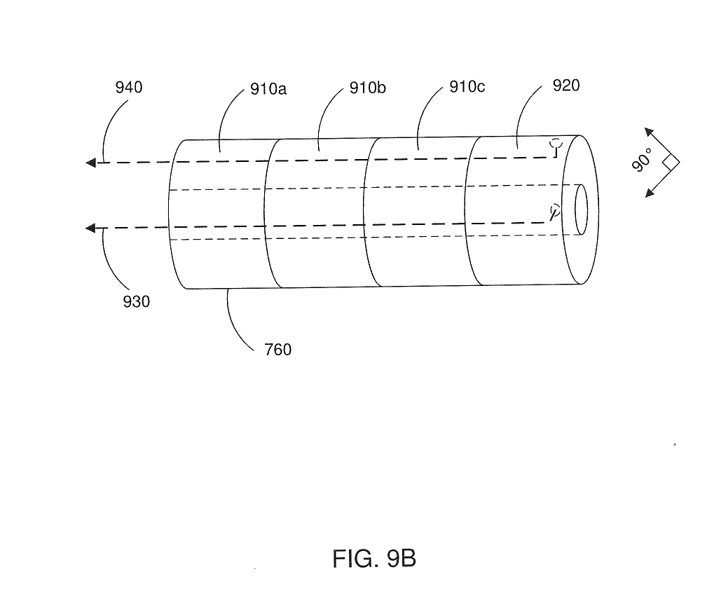

FIG. 9A illustrates distal tip 760 of hypotube 710. Navigation of the elongated shaft 110 is obtained by a combination of three actions, namely longitudinal movement of hypotube 710 by pushing from the proximal end, rotation of hypotube 710, and, finally, use of a navigation wire 930 (e.g., a tether). Navigation wire 930 can be fabricated from any high tensile material, such as stainless steel, para-aramid synthetic fiber (e.g., Kevlar®), and ultra-high-molecular-weight polyethylene (e.g., Dyneema®). Navigation wire 930 is anchored (e.g., by any appropriate anchoring means such as solder, welding, crimping, adhesives, knots and barbs) at the distal tip of hypotube 710 and runs the length of inner catheter 620 (see FIG. 6) back to handle 140 of articulated catheter 110 (see FIG. 1). As FIG. 9A illustrates, navigation wire 930 is located off-center with respect to the longitudinal axis of hypotube 710. Accordingly, when navigation wire 930 is pulled, distal tip 760 of hypotube 710 deflects towards the side of hypotube 710 to which navigation wire 930 is attached. Thus, distal tip 760 can be deflected radially outward from the longitudinal axis of the catheter, with navigation wire 930 controlling the degree of deflection. Rotation of distal tip 760 enables the radial deflection to be swept around 360 degrees. The anchor point of navigation wire 930 is shown at a location that is short of the end of distal tip 760. In a further embodiment, the anchor point of navigation wire 930 can be located as flush with the end of distal tip 760. The choice of an anchor point location that is short of the end of distal tip 760 leads to a non-deflectable straight section nearest the distal end of distal tip 760. During navigation, inner catheter 620 is typically flush or recessed within outer catheter 210. At the desired location within the body, inner catheter 620 can be extended in the direction provided by the deflected of distal tip 760 to provide the required medical procedure at the extended location. Thus, although outer catheter 210 is prevented from advancing through a tortuous pathway of diameter smaller than the diameter of outer catheter 210, inner catheter 210 can advance through the tortuous pathway if the diameter of inner catheter 210 is smaller than the diameter of the tortuous pathway. For example, the 5^{th} branch of an adult bronchial tree has a diameter of 3.5 mm. Thus, in an exemplary embodiment, outer catheter 210 with a diameter of 4.2 mm could not pass without potentially causing damage to a lumen or surrounding tissue, while inner catheter 620 with a diameter of 2.7 mm can pass along the 5^{th} branch.

FIG. 9B illustrates a further embodiment that provides two orthogonal directions of articulation. For example, in addition to navigation wire 930, a second navigation wire 940 can be disposed within one of the unused lumens 640 and attached to distal tip 760 at approximately 90 degrees to the attachment point of navigation wire 930 (see FIGS. 6, 7, 9A, 9B). By selectively pulling navigation wire 930 and/or second navigation wire 940, two orthogonal planes of deflection can be achieved. Navigation wires 930, 940 can be fabricated from any high tensile material, such as stainless steel, and anchored by any appropriate bonding means such as solder. In addition, anchor points of navigation wires 930, 940 can be either short of the end of distal tip 760 or flush with the end of distal tip 760. In a further embodiment, navigation wires 930, 940 can be attached to distal tip 760 at angles other than approximately 90 degrees to each other. For example, angles of 45, 60 and 180 degrees are within the scope of the present invention.

In one embodiment, distal tip 760 is formed from one or more coaxial segments 910a, 910b, 910c coupled together axially, with coaxial segment 910a coupled to distal end 750 of hypotube 710. Each segment is made of a material of a particular durometer, with its adjacent segment having a different durometer. Thus, for example, distal tip 760 can contain three segments 910a, 910b, 910c from proximal end to distal end, with durometers of 90, 20 and 60, respectively. Thus, by applying a force to navigation wire 930, distal tip 760 deflects, with the amount of deflection dictated by the sequence of durometer values in the segments in distal tip 760. Distal tip 760 therefore provides far greater articulability than that otherwise provided by an application of a longitudinal force to a simple shaft end. Each segment is bonded to its neighboring segment by any suitable bonding techniques, e.g., reflow techniques. Typical materials for manufacture of distal tip include polymers such as a thermoplastic elastomer such as Pebax®, and are typically the same material as that used for inner catheter 620, with differing durometers. Although the example illustrates three segments 910a, 910b, 910c, any number of segments 910 falls within the scope of the invention. Thus, by selecting the number of segments 910, the length of the segments 910 and the durometer of the segments 910, a wide variety of deflection angles are possible in the distal tip 760. Accordingly, difficult acute angles that are found, for example in a bronchial system, can be readily navigated by elongated device 100. In an exemplary embodiment, distal tip 760 contains three segments of length in the range 0.2 cm to 3 cm, and durometers in the range 20 to 70 Shore A.

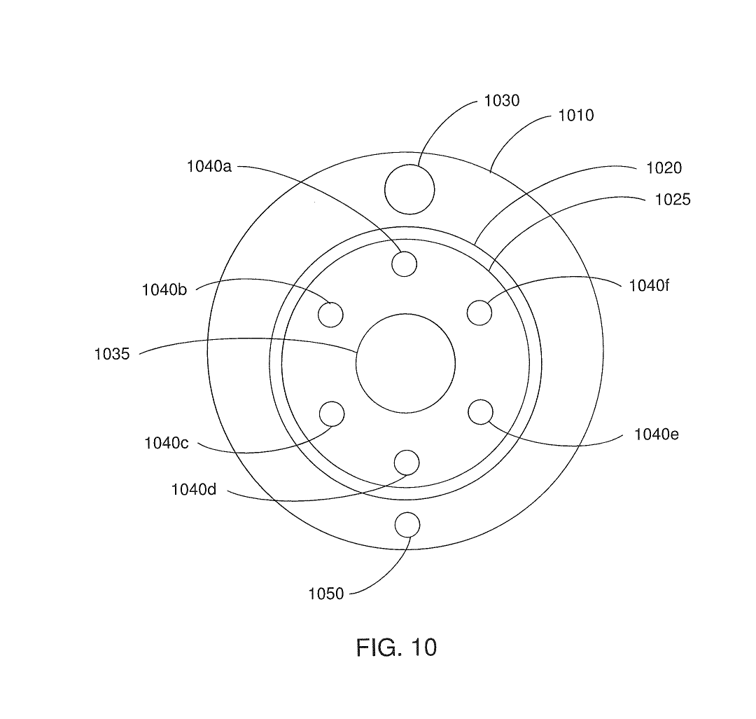

In a still further embodiment, FIG. 10 depicts a dual-catheter dual-articulation device, where the outer catheter 1010 and the inner catheter 1025 each have a navigation wire (not shown) fed through lumens 1050 and 1040a respectively. Thus, in this particular embodiment, articulation remains possible even when inner catheter 1025 has been removed from the interior of outer catheter 1010. By appropriate manual manipulation, lumens 1050 and 1040a can remain at an approximately 180 degree orientation to one another. Other orientations are within the scope of the present invention, by manual manipulation, by using alternative lumens 1040a through 1040f in inner catheter 1025, and by a different orientation by lumen 1050 in outer catheter 1010.

Embodiments of the present invention can be realized in the form of various endoscopes and other catheter-based devices to support medical procedures in pulmonology, cardiology, urology, gastroenterology and neurology, or any procedure involving a hollow organ. Access by the present invention to the desired site within the body can be by any natural orifice, small incision or through the use of any minimally invasive surgery in order to perform the desired task. Such access points include but are not limited to mouth, nose, urethra, and radial, jugular and femoral arteries. Lengths of the present invention range from 1 cm (as would be applicable in certain brain procedures), to a 5 cm length bronchoscope for use in a procedure on a small infant, to lengths in excess of 130 cm for use in various scopes such as endoscopes and bronchoscopes for adult procedures. Tools that can be attached to the present invention include a biopsy brush, biopsy forceps, an ablation needle, an advanced-energy tool and a coring tool. In an exemplary embodiment, a flexible bronchoscope can be realized. In a particular embodiment of the flexible bronchoscope, elongated shaft 110 would be about 62.5 to 125 cm (25 to 50 inches) long, with outer catheter 210 having a diameter of about 4.2 mm and containing two lumens 230, 320, having diameters of about 0.81 mm and 3.4 mm respectively. Lumen 230 supports the provision of a fiber-optic system, while lumen 320 supports the provision of inner catheter 620. Inner catheter 620 has a diameter of about 2.7 mm and supports at least two lumens 630, 640. Lumen 630 supports hypotube 710, while lumen 640 supports a navigation wire. The diameter of lumen 640 can be about 0.2 to 0.4 mm. Hypotube 710 has an outer diameter and an inner diameter of about 1.68 mm and 1.52 mm respectively.

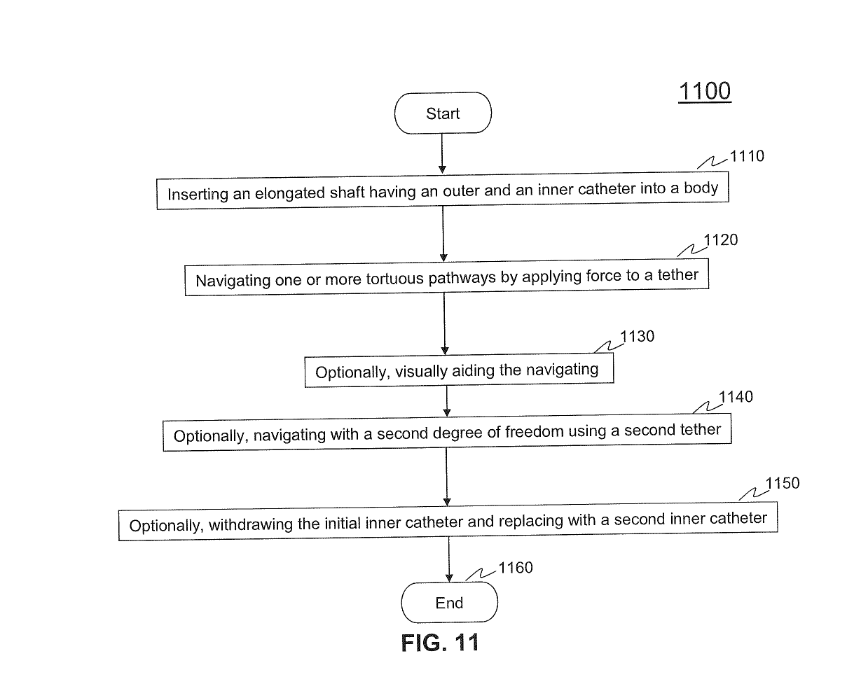

FIG. 11 provides a flowchart of an exemplary method 1100 to provide a method for navigating to a desired position within a body, according to an embodiment of the present invention.

The process begins at step 1110. In step 1110, an elongated shaft 110 having an outer catheter 210 and an inner catheter 220 is inserted into a body.

In step 1120, the elongated shaft 110 is navigated through a number of tortuous pathways within the body. For example, steerability can be achieved by applying a force to navigation wire 930 coupled to a distal end 760 attached to a hypotube 710 within the inner catheter 220 of elongated shaft 110.

In an optional or alternative step 1130, navigating can be visually aided by illumination (e.g., a fiber-optic light source) and vision system (e.g., a camera or lens arrangement) provided via an optical fiber 240 disposed within a fiber-optic lumen 230 within outer catheter 210.

In an optional or alternative step 1140, navigating over a second orthogonal range of motion. For example, steerability in a second direction can be provided by a second navigation wire attached to distal end 760 of hypotube 710 at approximately 90 degrees to the point of attachment of the navigation wire.

In an optional or alternative step 1150, after reaching desired location within the body, the inner catheter 220 can be withdrawn and a second inner catheter 220 can be inserted within the stationary outer catheter 210 to reach the desired location within the body.

At step 1160, method 1100 ends.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A dual-catheter apparatus comprising:
an outer catheter having at least one lumen and having sufficient flexibility to be advanced into a tortuous passageway;
an inner catheter disposed within a lumen of the outer catheter and having a proximal end and a distal end, wherein the distal end is configured to accept a tool; and
a navigation wire coupled to the distal end of the inner catheter, wherein manipulation of the navigation wire at the proximal end causes deflection of the distal end of the inner catheter, allowing steerability of the dual-catheter apparatus.

2. The apparatus of claim 1, wherein the lumen is eccentric with respect to the outer catheter.

3. The apparatus of claim 1, wherein a cross-section of the lumen is an extended circle configured to resist shrinkage within the outer catheter.

4. The apparatus of claim 1, further comprising:
a vision delivery system disposed within a further lumen of the outer catheter.

5. The apparatus of claim 4, wherein the vision delivery system includes a fiber-optic line.

6. The apparatus of claim 5, wherein the vision delivery system is re-usable.

7. The apparatus of claim 1, wherein a diameter of the outer catheter is less than 5 mm.

8. The apparatus of claim 1, wherein an outer surface of the inner catheter is coated with a hydrophilic layer.

9. The apparatus of claim 1, wherein the inner catheter is configured to be replaceable with another inner catheter while the outer catheter remains substantially stationary in a body.

10. The apparatus of claim 1, wherein the tool is one of a diagnostic tool and a therapeutic tool.

11. The apparatus of claim 1, wherein the inner catheter comprises:
a hypotube having a longitudinal axis concentric with a longitudinal axis of the inner catheter.

12. The apparatus of claim 11, wherein the hypotube further comprises:
a plurality of transverse slots fabricated at a distal end of the hypotube.

13. The apparatus of claim 12, wherein a frequency of the plurality of the transverse slots varies along a direction of the longitudinal axis.

14. The apparatus of claim 11, further comprising:
a distal tip coupled to the distal end of the inner catheter and having an attachment point to which the navigation wire is attached, wherein the distal tip is formed from one or more adjoining segments.

15. The apparatus of claim 14, wherein the distal tip is formed from a plurality of adjoining segments, and wherein at least two of the segments are formed from materials having differing durometers.

16. The apparatus of claim 1, further comprising:
a further navigation wire coupled to a point on the distal end of the inner catheter that is orthogonal to a coupling point of the first navigation wire, wherein manipulation of the further navigation wire at the proximal end causes an orthogonal deflection of the distal end of the inner catheter, allowing additional steerability of the dual-catheter apparatus.

17. The apparatus of claim 1, wherein the inner catheter further comprises a plurality of lumens, each lumen being configured to receive a further navigation wire or connectivity to a tool, or to deliver or receive a tissue or fluid.

18. A dual-catheter apparatus comprising:
an outer catheter having a distal end and a proximal end, a first lumen and a second lumen, and having sufficient flexibility to be advanced into a tortuous passageway;
an inner catheter disposed within the first lumen of the outer catheter and having a proximal end and a distal end, wherein the distal end of the inner catheter is configured to accept a tool;
a first navigation wire disposed in the second lumen of the outer catheter and coupled to the distal end of the outer catheter; and
a second navigation wire coupled to the distal end of the inner catheter, wherein manipulation of the first navigation wire at the proximal end of the outer catheter and manipulation of the second navigation wire at the proximal end of the inner catheter causes deflection of the distal end of the outer catheter and the inner catheter respectively, allowing steerability of the dual-catheter apparatus.

19. A method comprising:
inserting an elongated medical device having an outer catheter and an inner catheter into a tortuous passageway in a body, wherein the outer catheter has at least one lumen and has sufficient flexibility to be advanced into the tortuous passageway, the inner catheter being disposed within a lumen of the outer catheter and having a proximal end and a distal end, the distal end being configured to accept a tool; and
navigating the elongated shaft along the tortuous pathway by manipulating a navigation wire coupled to the distal end of the inner catheter, wherein manipulation of the navigation wire at the proximal end causes deflection of the distal end of the inner catheter.

20. The method of claim 19, further comprising:
illuminating an environment surrounding the distal end of the inner catheter by using fiber optics connected to a fiber-optic line disposed within a further lumen in the outer catheter.

21. The method of claim 19, further comprising:
navigating the elongated shaft by manipulating a further navigation wire coupled to a point on the distal end of the inner catheter that is orthogonal to a connection point of the navigation wire.

22. The method of claim 19, further comprising:
replacing the inner catheter with a further inner catheter while maintaining the outer catheter in a substantially stationary position in a body.

23. A bronchoscope comprising:
an outer catheter having at least one lumen, an outer diameter of less than about 5 mm, and sufficient flexibility to be advanced into a tortuous passageway in a bronchial system, wherein a length of the outer catheter is in the range of about 62.5 cm to 125 cm;
an inner catheter disposed within a lumen of the outer catheter and having a proximal end and a distal end, wherein the distal end is configured to accept a tool and wherein the inner catheter includes a hypotube made from stainless steel, having an outer diameter of about 1.68 mm and a pattern of transverse slots to provide flexibility; and
a navigation wire coupled to the distal end of the inner catheter, wherein manipulation of the navigation wire at the proximal end causes deflection of the distal end of the inner catheter, allowing steerability of the bronchoscope.

24. The bronchoscope of claim 23, wherein the inner catheter further comprises:
a hypotube having a longitudinal axis concentric with a longitudinal axis of the inner catheter, the hypotube having a plurality of transverse slots fabricated at a distal end of the hypotube, and
a distal tip coupled to the distal end of the hypotube, wherein the distal tip is formed form a plurality of adjoining segments, and wherein at least two of the segments are formed from materials having differing durometers.
